(19) United States
(12) Patent Application Publication
(10) Pub. No.: US 2025/0034359 A1
(43) Pub. Date: Jan. 30, 2025

(54) CROP-BASED PLASTICIZERS

(71) Applicant: The United States of America, as Represented by the Secretary of Agriculture, Washington, DC (US)

(72) Inventor: MICHAEL A. JACKSON, MORTON, IL (US)

(21) Appl. No.: 18/360,561

(22) Filed: Jul. 27, 2023

(51) Int. Cl. C08K 5/1535; C07D 307/14

(52) U.S. Cl. CPC C08K 5/1535; C07D 307/14

(57) ABSTRACT

Disclosed are plasticizer compositions in the form of crop-based secondary amines prepared through catalyzed reductive amination (RA) of furans with aminated ketones. Exemplary furans include, but are not limited to, furfural, 5-(hydroxymethyl) furfural (HMF), and 2,5-diformylfuran (DFF). These furans may be crop-based. Furfural, for example, may be made through dehydration of xylose isolated from corn cobs. Exemplary aminated ketones include, but are not limited to, crop-based aminated ketones such as 2-aminoundecane. For example, 2-aminoundecane may be prepared through catalyzed RA of 2-undecanone from the seed oil of Cuphea. These RA reactions may be catalyzed using, for example, Pd/C. In some embodiments, the plasticizer compositions function as plasticizers when blended with polyvinylchloride (PVC) and/or other polymers to form plasticized compositions. Processes for making the plasticizer compositions and plasticized compositions are also disclosed. The plasticizer compositions provide a sustainable and comparable alternative to known petroleum-based phthalate ester plasticizers, such as diethylhexyl phthalate (DEHP).

### BACKGROUND OF THE INVENTION

Disclosed are plasticizer compositions in the form of crop-based secondary amines prepared through catalyzed reductive amination (RA) of furans with aminated ketones. Exemplary furans include, but are not limited to, furfural, 5-(hydroxymethyl) furfural (HMF), and 2,5-diformylfuran (DFF). These furans may be crop-based. Furfural, for example, may be made through dehydration of xylose isolated from corn cobs. Exemplary aminated ketones include, but are not limited to, crop-based aminated ketones such as 2-aminoundecane. For example, 2-aminoundecane may be prepared through catalyzed RA of 2-undecanone from the seed oil of Cuphea. These RA reactions may be catalyzed using, for example, Pd/C. In one or more embodiments, the plasticizer compositions function as plasticizers when blended with polyvinylchloride (PVC) and/or other polymers to form plasticized compositions. Processes for making the plasticizer compositions and plasticized compositions are also disclosed. The plasticizer compositions provide a sustainable and comparable alternative to known petroleum-based phthalate ester plasticizers, such as diethylhexyl phthalate (DEHP).

The ubiquity of plastics has accelerated economic growth in the past century by enabling new technologies, reducing energy use by reducing the weight of vehicles and containers, reducing food waste by extending the shelf life of foods, and making medical treatment safer through single-use supplies. One of the most common plastics with the most diversified uses is poly(vinylchloride), or PVC. Known colloquially as “vinyl,” it can be found in long-lasting construction materials such as plumbing, electrical insulation, flooring, siding, and windows. It is common in cars in underbody coatings, wire harnesses, and throughout the passenger compartment in dashboards, steering wheels, and armrests. It is also used in disposable items such as blood bags, credit cards, and toys. The broad use of PVC is attributable to its versatility, and this is possible due to the ease with which PVC can be blended with modifiers such as pigments, air-release agents, fillers, flame retardants, and especially, plasticizers.

Plasticizers are defined by the International Union of Pure and Applied Chemistry as a material that is added to another material to add flexibility, workability, and distensibility. Thus, plasticizers are added to polymers to impart flexibility and processability to otherwise rigid and brittle plastics. (Godwin, A. D., Chapter 24: Plasticizers, in Applied Plastics Engineering Handbook: Processing, Materials, and Applications (Second Edition), Kutz, M., Ed., William Andrew Publishing, 533-553 (2016)). The most commonly used plasticizer for PVC, accounting for about 85% of the market, is bis(2-ethylhexyl) phthalate (DEHP). This compound has come under scrutiny, however, for potential adverse health impacts, particularly as an endocrine disrupter (Lyche, J. L., et al., J Toxicol Environ Health B Crit Rev, 12 (4): 225-249 (2009); Busgang, S. A., et al., Sci. Total Environ, 850:157830 (2022); Rebuzzini, P., et al., Cells, 11 (19): 3163-3170 (2022); Eales, J., et al., Environ Int., 158:106903 (2022)), as well as an environmental pollutant (Das, M. T., et al., J. Hazard. Mater., 409:124496 (2021)). The safety of DEHP is of concern so a biobased substitute is attractive especially for things like soft toys, milk bottle nipples, and dog chew toys.

Consequently, there has been a recent push to develop biobased plasticizers as a replacement for DEHP (Arias, K. S., et al., ChemSusChem, 13 (7): 1864-1875 (2020); Chen, J., et al., ACS Sustainable Chem. Eng., 6 (1): 642-651 (2018); Elsiwi, B. M., et al., ACS Sustainable Chem. Eng., 8 (33): 12409-12418 (2020); Erythropel, H. C., et al., Polymer, 89:18-27 (2016); Ortega-Toro, R., et al., Heliyon, 7 (2): e06176 (2021); van Vugt-Lussenburg, B. M. A., et al., Green Chem., 22 (6): 1873-1883 (2020); Yang, Y, et al., Materials & Design, 126:29-36 (2017); Zhang, H., et al., Polym. Test., 76:199-206 (2019); Zhu, H., et al., ACS Sustainable Chem. Eng., 9 (45): 15322-15330 (2021); Viera, M. G. A., et al., Eur. Polym. J., 47 (3): 254-263 (2011); Bocqué, M., et al., J. Polym. Sci., Part A: Polym. Chem., 54 (1): 11-33 (2016); Greco, A., et al., Polym. Degrad. Stab., 95 (11): 2169-2174 (2010); Halloran, M. W., et al., J. Appl. Polym. Sci., 139 (32): e52778 (2022); He, Z., et al., Polym. Test., 91:106793 (2020); Park, M., et al., Journal of Industrial and Engineering Chemistry, 88:148-158 (2020); Rigotti, D., et al., ACS Sustainable Chem. Eng., 9 (41): 13742-13750 (2021); Fenollar, O., et al., J. Mater. Sci., 44 (14): 3702-3711 (2009)). These new compounds have been synthesized based on various sustainable sources including seed oils (Chen et al., ibid; Ortega-Toro et al., ibid; Fenollar et al., ibid), fatty acid esters, citric, succinic and malic acid derivatives (Park et al., ibid; Erythropel, H. C., et al., Chemosphere, 91 (3): 358-365 (2013); Umemura, R. T., et al., J. Appl. Polym. Sci., 138 (10): 49990 (2021)), sugar and glycerol derivatives (Arias et al., ibid; Yang et al., ibid; Halloran et al., ibid; He et al., ibid; Rigotti et al., ibid), cardanol from cashew nuts (Greco et al., ibid), and vanillic acid that can be sourced from lignin (Zhu et al., ibid). In addition to circumventing any health and environmental impacts from DEHP, biobased plasticizers can improve the sustainability metrics of PVC. The high chlorine content of PVC results in relatively low carbon content, all of which is currently petroleum-based and not sustainable. Therefore, the addition of crop-based plasticizer at the common concentrations results in meaningful improvement in these metrics.

Cuphea is a genus of annual and perennial plants native to the Americas. It is under development as a new row crop owing to its high concentration of medium chain-length fatty acids in its seed oil (Graham, S. A., et al., Amer. J. Bot., 68 (7): 908-917 (1981)). This makes Cuphea a promising substitute for palm and coconut oils that can be grown on marginal lands in temperate regions. To advance development of a new crop, new uses that will drive demand for the crop are required. To this end, previous work in our lab with Cuphea seed oil has resulted in the preparation of 2-undecanone (Jackson, M. A., et al., Appl. Catal. A: Gen., Vols. 431-432:157-163 (2012)). This compound is used as a fragrance and is also known as an effective mosquito and tick repellent. Furthermore, this long-chain ketone has been used as a substrate in a reductive amination reaction with sugar C-glycoside amines (Jackson, M. A., et al., ACS Sustainable Chem. Eng., 9 (41), 13842-13850 (2021)) resulting in a new family of biobased surfactants. In this disclosure, we describe further chemistry using crop-sourced 2-undecanone to prepare compounds that perform well as plasticizers for PVC. These compounds are a priori less toxic and have shorter lifetimes in the environment.

### SUMMARY OF THE INVENTION

Disclosed are plasticizer compositions in the form of crop-based secondary amines prepared through catalyzed reductive amination (RA) of furans with aminated ketones. Exemplary furans include, but are not limited to, furfural, 5-(hydroxymethyl) furfural (HMF), and 2,5-diformylfuran (DFF). These furans may be crop-based. Furfural, for example, may be made through dehydration of xylose isolated from corn cobs. Exemplary aminated ketones include, but are not limited to, crop-based aminated ketones such as 2-aminoundecane. For example, 2-aminoundecane may be prepared through catalyzed RA of 2-undecanone from the seed oil of Cuphea. These RA reactions may be catalyzed using, for example, Pd/C. In one or more embodiments, the plasticizer compositions function as plasticizers when blended with polyvinylchloride (PVC) and/or other polymers to form plasticized compositions. Processes for making the plasticizer compositions and plasticized compositions are also disclosed. The plasticizer compositions provide a sustainable and comparable alternative to known petroleum-based phthalate ester plasticizers, such as diethylhexyl phthalate (DEHP).

An exemplary series of crop-based secondary amines that function as plasticizers was prepared, in accordance with one or more embodiments, through the reductive amination (RA) of furans with 2-aminoundecane (2-AUD) using Pd/C as the catalyst for the reaction. The 2-AUD was itself prepared through the RA of 2-undecanone that can be prepared from the seed oil of Cuphea. The 2-AUD was added to furfural, 5-(hydroxymethyl) furfural (HMF), and 2,5-diformylfurfural (DFF) in a second RA to give the plasticizers. The plasticizers were blended at 22 wt % with poly(vinylchloride) and films of the blends were cast onto glass plates. The films were evaluated by mechanical properties, scanning electron microscopy (SEM), atomic force microscopy (AFM), microscope infrared spectroscopy, UV transmittance, dynamic mechanical analysis (DMA), and thermogravimetric analysis (TGA). These results were compared to films made with di(2-ethylhexyl) phthalate and films made without plasticizer. The results suggest that these biobased plasticizers produce a flexible film comparable to that made with the petroleum-based DEHP.

This summary is provided to introduce a selection of concepts in a simplified form that are further described below in the detailed description. This summary is not intended to identify key features of the claimed subject matter, nor is intended as an aid in determining the scope of the claimed invention.

### BRIEF DESCRIPTION OF THE SEVERAL VIEWS OF THE DRAWINGS

Embodiments will hereinafter be described in conjunction with the appended drawings, where like designations denote like elements.

FIG. 1 depicts UV/vis spectra in transmittance mode of a PVC control film, a PVC benchmark film with DEHP plasticizer, and exemplary PVC films with THF-2-AUD, HMF-2-AUD, and DFF-2-AUD plasticizers, respectively, according to one or more embodiments.

FIG. 2 depicts FT-IR spectra from 600 to 3550 cm-1 of the pure DEHP plasticizer, and exemplary pure THF-2-AUD, HMF-2-AUD, and DFF-2-AUD plasticizers, respectively, according to one or more embodiments.

FIG. 3 depicts FT-IR spectra from 650 to 1800 cm-1 of a PVC control film, a PVC benchmark film with DEHP plasticizer, and exemplary PVC films with THF-2-AUD, HMF-2-AUD, and DFF-2-AUD plasticizers, respectively, according to one or more embodiments.

FIG. 4 depicts FT-IR spectra from 2700 to 3550 cm-1 of a PVC control film, a PVC benchmark film with DEHP plasticizer, and exemplary PVC films with THF-2-AUD, HMF-2-AUD, and DFF-2-AUD plasticizers, respectively, according to one or more embodiments.

FIGS. 5A-5D depict DMA curves for a PVC benchmark film with DEHP plasticizer and exemplary PVC films with THF-2-AUD, HMF-2-AUD, and DFF-2-AUD plasticizers, respectively, according to one or more embodiments. FIG. 5A plots the change in length curve, FIG. 5B plots the tan 8 curve, FIG. 5C plots the loss modulus curve, and FIG. 5D plots the storage modulus curve. The key (legend) shown and used in FIG. 5A is also used (but not shown) in FIGS. 5B-5D.

FIGS. 6A-6C depict TGA curves for a PVC benchmark film with DEHP plasticizer and exemplary PVC films with THF-2-AUD, HMF-2-AUD, and DFF-2-AUD plasticizers, respectively, according to one or more embodiments. FIG. 6A plots the mass loss curve, FIG. 6B plots the first derivative mass loss curve, and FIG. 6C plots the HCl evolution curve. The key (legend) shown and used in FIG. 6A is also used (but not shown) in FIG. 6B; whereas, a slightly different key (legend) is shown and used in FIG. 6C that adds a thick black line to denote the HCl evolution curve for the PVC control film (i.e., without plasticizer).

### DETAILED DESCRIPTION

Disclosed are plasticizer compositions in the form of crop-based secondary amines prepared through catalyzed reductive amination (RA) of furans with aminated ketones. Exemplary furans include, but are not limited to, furfural, 5-(hydroxymethyl) furfural (HMF), and 2,5-diformylfuran (DFF). These furans may be crop-based. Furfural, for example, may be made through dehydration of xylose isolated from corn cobs. Exemplary aminated ketones include, but are not limited to, crop-based aminated ketones such as 2-aminoundecane. For example, 2-aminoundecane may be prepared through catalyzed RA of 2-undecanone from the seed oil of Cuphea. These RA reactions may be catalyzed using, for example, Pd/C. In one or more embodiments, the plasticizer compositions function as plasticizers when blended with polyvinylchloride (PVC) and/or other polymers to form plasticized compositions. Processes for making the plasticizer compositions and plasticized compositions are also provided. The plasticizer compositions provide a sustainable and comparable alternative to known petroleum-based phthalate ester plasticizers, such as diethylhexyl phthalate (DEHP).

As noted above, exemplary furans include, but are not limited to, furfural, 5-(hydroxymethyl) furfural (HMF), and 2,5-diformylfuran (DFF). These furans may be crop-based. Furfural, for example, may be made through dehydration of xylose isolated from corn cobs. Furfural, HMF, and DFF are commercially available. One of ordinary skill in the art will appreciate, however, that other crop-based furans and non-crop-based furans may be utilized in accordance with one or more embodiments of the present invention in lieu of, or in addition to, furfural, HMF, and/or DFF.

As also noted above, exemplary aminated ketones include, but are not limited to, crop-based aminated ketones such as 2-aminoundecane. For example, 2-aminoundecane may be prepared through catalyzed RA of 2-undecanone derived from the seed oil of Cuphea. This catalyzed RA is between, for example, 2-undecanone (and/or one or more other ketone, as described herein) and NH₃. Generally, these reactions are performed in a solvent (e.g., 1000 ml MeOH) in which the 2-undecanone (e.g., 150 g) (and/or one or more other ketone) is soluble (e.g., an organic solvent such as methanol, ethanol, or the like), and are catalyzed by a hydrogenation catalyst (e.g., 2 g, 5 wt % Pd/C (palladium on any support will work, in addition to platinum and rhodium)). The reactor is purged of air with hydrogen or an inert gas (nitrogen or argon) prior to the addition of NH₃ at about 1.5 to about 3.1 bar (e.g., 1.5-3.1 bar) anhydrous, and then heated to about 65° C. to about 100° C. (e.g., 65° C. to 100° C.). The reactor is then charged to about 17 bar to about 70 bar (e.g., 17-70 bar) with H₂. For example, the reaction may be performed under 34 bar hydrogen (H₂) pressure with the reactor heated to 80° C. Reaction progress is monitored by GS/MS (gas chromatography mass spectrometry), MALDI-TOF (matrix-assisted laser desorption/ionization-time-of-flight) mass spectrometry, or the like. Upon completion (generally about 1 h to about 6 h (e.g., 1-6 h)). The catalyst is removed by filtration and the filtrate (product) is taken to dryness using a rotary evaporator. The RA reaction may be performed using, for example, palladium, platinum, and/or rhodium as the active metal. Effective supports include, for example, Al₂O₃, HMS-SiO₂, C, and the zeolites ZSM-5, beta, and mordenite.

The reductive amination of ketones with NH₃ generally leads to secondary amines since primary amines are more reactive than is NH₃ (Nakamura, Y., et al., ChemCatChem, 7:921-924 (2015)). We surprisingly solved this potential problem by having about 10 to about 25 fold (e.g., 10-25 fold) molar excess (compared to the 2-undecanone) of NH₃ in the reactor.

Alternatively, 2-aminoundecane is commercially available. In accordance with one or more embodiments of the present invention, 2-aminoundecane may be purchased commercially in lieu of being prepared through catalyzed RA of 2-undecanone as described herein.

Moreover, one of ordinary skill in the art will appreciate that other crop-based amines and non-crop-based amines may be utilized in accordance with one or more embodiments of the present invention in lieu of, or in addition to, 2-aminoundecane. Other exemplary amines include, but are not limited to, 1-amino alkanes such as decylamine, tetradecylamine, and hexadecylamine, as well as other 2-amino alkanes such as 2-aminotridecane. Each of these other exemplary amines is commercially available.

As noted above, 2-undecanone may be prepared from the seed oil of Cuphea. Cuphea seed oil may be uniquely suited to the cross ketonization reaction with acetic acid (Jackson, M. A., et al., Appl. Catal. A: Gen., Vols. 431-432:157-163 (2012)). This condensation reaction converts two carboxylic acids to a ketone with the elimination of CO₂ and water. For example, two acetic acid molecules react to form acetone. The fatty acid composition of Cuphea seed triacylglyceride is typically about 72% decanoic acid. In the ketonization reaction with acetic acid, the decanoic acid is converted to 2-undecanone at 90% yield. This is high temperature chemistry performed in a flow reactor. Alternatively, 2-undecane is commercially available.

Other exemplary aminated ketones include, but are not limited to, aminated ketones prepared through catalyzed RA of any other C3-C22 ketone ranging from, for example, 2-pentanone to 2-nonadecanone and 10-nonadecanone, as well as 3-dodecanone, 2-tridecanone, 2-pentadecanone, and 2-heptadecanone. Each of these ketones is commercially available. Like 2-undecanone, 3-dodecanone may be prepared from the seed oil of Cuphea. Other possible long-chain ketones may be prepared from other plant oils, for example, soy or corn oils.

While a variety of plant oils may be used in the condensation reaction for the production of ketones, preferred oils are those comprising a relatively high proportion of esterified decanoic acid, particularly (′uphea, palm, and coconut oil, with Cuphea being particularly preferred. The oils comprising esterified short chain fatty acids such as decanoic acid, are relatively volatile in comparison to other oils, rendering them particularly suited for a gas phase ketonization reaction. As noted above, the fatty acid composition of Cuphea seed triacylglyceride is typically about 72% decanoic acid. In the ketonization reaction with propionic acid, the decanoic acid is converted to 3-dodecanone. The resulting 3-dodecanone may be aminated through catalyzed RA under conditions similar to those described herein with respect to the catalyzed RA of 2-undecanone.

General novel methods for the preparation of aminated furan-based plasticizers: This catalyzed RA is between, for example, 2-aminoundecanone (and/or other crop-based aminated ketone and/or non-crop-based aminated ketone) and a furan (e.g., THF, HMF, DFF, other crop-based furan, and/or non-crop-based furan). These reactions are reductive aminations between the aldehyde carbonyl of the furans with the 2-AUD, for example, acting as the amine. The resulting Schiff's base is subsequently hydrogenated using a hydrogenation catalyst, such as Pd/C, under an atmosphere of H₂. Generally, these reactions are performed in a solvent in which the aminated ketone and the furan are soluble (e.g., an organic solvent such as methanol, ethanol, or the like), and are catalyzed by a hydrogenation catalyst (e.g., Pd/C (palladium on any support will work, in addition to platinum and rhodium)). In a 300 ml high-pressure reactor (e.g., Parr Instruments, Inc.), for example, a furan (e.g., THF, HMF, DFF, other crop-based furan, and/or non-crop-based furan, preferably freshly distilled) and a 10% molar excess of, for example, 2-aminoundecane (and/or other crop-based aminated ketone and/or non-crop-based aminated ketone) are dissolved in a solvent (e.g., 40 ml MeOH) along with the heterogeneous catalyst (e.g., 300 mg, 5 wt % Pd/C). The closed reactor vessel is then purged of air with H₂ or an inert gas (nitrogen or argon), heated to about 65° C. to about 100° C. (e.g., 65° C. to 100° C.), and finally charged to about 17 bar to about 70 bar (e.g., 17-70 bar) H₂. For example, the reaction may be performed under 34 bar hydrogen (H₂) pressure with the reactor heated to 80° C. Reaction progress is monitored by GS/MS (gas chromatography mass spectrometry), MALDI-TOF (matrix-assisted laser desorption/ionization-time-of-flight) mass spectrometry, or the like. Upon completion (generally about 2 h to about 4 h (e.g., 2-4 h)). The catalyst is removed by filtration and the filtrate (product) is taken to dryness (i.e., the solvent removed) using a rotary evaporator. The aminated furan-based plasticizer product (e.g., THF-2-AUD, HMF-2-AUD, and the like) may be collected by short path vacuum distillation, though DFF-2-AUD could not be distilled. The RA reaction may be performed using, for example, palladium, platinum, and/or rhodium as the active metal. Effective supports include, for example, Al₂O₃, HMS-SiO₂, C, and the zeolites ZSM-5, beta, and mordenite.

As noted above, these reactions are reductive aminations between the aldehyde carbonyl of the furans with the 2-AUD, for example, acting as the amine. The resulting Schiff's base is subsequently hydrogenated using a hydrogenation catalyst, such as Pd/C, under an atmosphere of H₂. This also results in the hydrogenation of the furan ring to a tetrahydrofuran moiety giving fully saturated products, which is beneficial since this results in colorless final products-thereby making the plasticizer colorless and unlikely to “yellow” after being blended. In fact, the exemplary PVC films produced here remained colorless.

General novel methods for the preparation a polymer film plasticized with an aminated furan-based plasticizer: An aminated furan-based plasticizer is blended at a suitable mass fraction (e.g., 22 wt %) with a polymer (e.g., poly(vinylchloride)) and films of the blends may be, for example, cast onto glass plates. For example, a PVC film is typically prepared with about 10 wt % to about 30 wt % (e.g., 10-30 wt %) (preferably 22 wt %) aminated furan-based plasticizer. PVC films plasticized with aminated furan-based plasticizer were prepared by casting tetrahydrofuran (THF) solutions of poly(vinyl) chloride (e.g., average MW 62,000 Da) onto glass Petri dishes. Typically, PVC (e.g., 200 mg) was stirred in THF (e.g., 5 ml) until a homogenous solution was obtained. Then, the aminated furan-based plasticizer (e.g., 32 mg) was added to this solution. The cast films were allowed to dry in a fume hood (e.g., 24 h) and then further dried in a vacuum oven at ambient temperature (e.g., 48 h).

One of ordinary skill in the art will appreciate, however, that the plasticized composition (i.e., a composition containing a polymer blended with an aminated furan-based plasticizer) may be processed using other known plastics processing techniques in lieu of, or in addition to, casting. For example, the plasticized composition may be processed using known plastics processing techniques such as injection molding, blow molding, thermoforming, transfer molding, reaction injection molding, compression molding, extrusion, and the like.

Moreover, in accordance with one or more embodiments of the invention, the plasticized composition contains at least one polymer blended with at least one aminated furan-based plasticizer, and, optionally, at least one known plasticizer such as DEHP.

For example, the plasticized composition may include at least one polymer including, but not limited to, polyvinyl chloride (PVC), polylactic acid (PLA), cellulose acetate, nitrocellulose, cellulose acetate butyrate (CAB), alkyd resins, acrylic resins, nylon, polystyrene, polyurethanes, ethyl cellulose, polyvinyl acetate phthalate (PVAP), cellulose acetate phthalate (CAP), nitrile butadiene rubber (NBR) (known colloquially as “nitrile rubber”), polychloroprene (known colloquially as “neoprene”), ethylene propylene diene monomer (EPDM) rubber, chlorinated polyethylene, and polymers derived from epichlorohydrin.

Plasticizers work by disrupting the matrix of the polymer. This happens through van Der Waals interaction and hydrogen bonding. The latter occur between the heteroatoms in plasticizers and the polarized H—C—Cl bond in PVC. THF-2-AUD has an oxygen atom in the tetrahydrofuran ring as well as the amine group beta to the ring. A nitrogen atom is novel when looking at the common plasticizers. This may offer particularly strong hydrogen bonding. The HMF-2-AUD has these features as well as a second oxygen atom just off the tetrahydrofuran ring. The DFF-2-AUD includes an additional amine group-one amine group on each beta-position of the tetrahydrofuran ring.

Other compounds (e.g., plasticizers known in the art) may be added to the composition provided they do not substantially interfere with the intended activity and efficacy of the composition; whether or not a compound interferes with activity and/or efficacy can be determined, for example, by the procedures utilized below.

“Optional” or “optionally” means that the subsequently described event or circumstance may or may not occur, and that the description includes instances in which said event or circumstance occurs and instances where it does not. For example, the phrase “optionally comprising a known plasticizer” means that the composition may or may not contain a known plasticizer and that this description includes compositions that contain and do not contain a known plasticizer. Also, by example, the phrase “optionally adding a known plasticizer” means that the method may or may not involve adding a known plasticizer and that this description includes methods that involve and do not involve adding a known plasticizer.

By the term “effective amount” of a compound or property as provided herein is meant such amount as is capable of performing the function of the compound or property for which an effective amount is expressed. As will be pointed out below, the exact amount required will vary from process to process, depending on recognized variables such as the compounds employed and the processing conditions observed. Thus, it is not possible to specify an exact “effective amount.” However, an appropriate effective amount may be determined by one of ordinary skill in the art using only routine experimentation.

While this invention may be embodied in many different forms, there are described in detail herein specific preferred embodiments of the invention. The present disclosure is an exemplification of the principles of the invention and is not intended to limit the invention to the particular embodiments illustrated. All patents, patent applications, scientific papers, and any other referenced materials mentioned herein are incorporated by reference in their entirety. Furthermore, the invention encompasses any possible combination of some or all of the various embodiments and characteristics described herein and/or incorporated herein. In addition, the invention encompasses any possible combination that also specifically excludes any one or some of the various embodiments and characteristics described herein and/or incorporated herein.

The amounts, percentages and ranges disclosed herein are not meant to be limiting, and increments between the recited amounts, percentages and ranges are specifically envisioned as part of the invention. All ranges and parameters disclosed herein are understood to encompass any and all subranges subsumed therein, and every number between the endpoints. For example, a stated range of “1 to 10” should be considered to include any and all subranges between (and inclusive of) the minimum value of 1 and the maximum value of 10 including all integer values and decimal values; that is, all subranges beginning with a minimum value of 1 or more, (e.g., 1 to 6.1), and ending with a maximum value of 10 or less, (e.g. 2.3 to 9.4, 3 to 8, 4 to 7), and finally to each number 1, 2, 3, 4, 5, 6, 7, 8, 9, and 10 contained within the range.

Unless otherwise indicated, all numbers expressing quantities of ingredients, properties such as molecular weight, reaction conditions (e.g., reaction time, temperature), percentages and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. As used herein, the term “about” refers to a quantity, level, value, or amount that varies by as much as 10% to a reference quantity, level, value, or amount. For example, about 1.0 g means 0.9 g to 1.1 g and all values within that range, whether specifically stated or not.

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described.

The following examples are intended only to further illustrate the invention and are not intended to limit the scope of the invention as defined by the claims.

### EXAMPLES

Preparation of the plasticizers. Commercial 2-undecanone was converted to 2-aminoundecane (2-AUD) through a reductive amination (RA) reaction using 5 wt % Pd/C as the hydrogenation catalyst. This conversion is accomplished at high yield by using a large excess of ammonia, thereby preventing the formation of the favored secondary amine. Scheme 1 shows the reactions of 2-AUD with the furan substrates. These reactions are reductive aminations between the aldehyde carbonyl of the furans with the 2-AUD acting as the amine. The resulting Schiff's base in subsequently hydrogenated using Pd/C catalyst under an atmosphere of H₂. This also results in the hydrogenation of the furan ring giving fully saturated products, which is beneficial since this results in colorless final products.

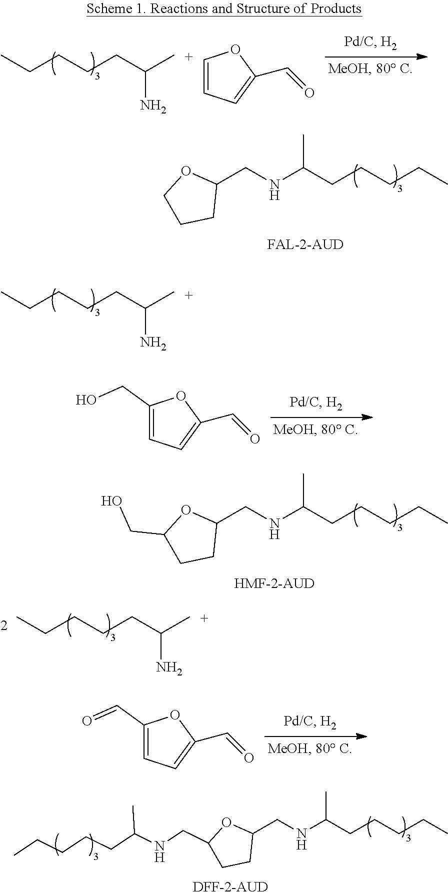

Preparation of the PVC films. The PVC films were prepared by casting a tetrahydrofuran (THF) solution of poly(vinylchloride) onto glass Petri dishes. The plasticized films also contained 22 wt % plasticizer, a loading that is comparable to that used by other researchers. Rogalsky, S., et al., J. Mater. Sci., 57 (10): 6102-6114 (2022). The resulting films had a glass-side and an air-side that were imaged by atomic force microscopy (AFM). For the purposes of this disclosure, the control film is PVC film without added plasticizer and the benchmark film is PVC film prepared with 22 wt % DEHP.

Mechanical Properties of the PVC films. The mechanical properties of plasticized PVC can be used to establish the best uses for the products. Therefore, tensile strength, elongation, and modulus were investigated, and these results are shown in Table 2. The plasticizers all contribute to lessening the rigidity and brittleness of the produced films relative to the control PVC film.

Surface Features of the PVC films. The surface of the PVC control film is relatively free of defects, but it does have small holes of approximately 0.5 to 1 μm in the film. When DEHP was added as plasticizer, somewhat larger holes developed in the film. No other surface features are observed in the film by SEM examination. For the film plasticized with THF-2-AUD, the small holes were again observed, however, there were also scattered particles on the surface of the film that were a few microns across. The HMF-2-AUD plasticized film displayed particles and cubic (up to 5×10 μm) materials on the film surface. This suggests that the HMF-2-AUD may not be fully compatible with the PVC. The DFF-2-AUD plasticized films also had particles (0.1-4.0 μm long axis) and 3-30 μm wide depressions, which suggests that the DFF-2-AUD is not fully miscible with PVC. The surfaces of the HMF-2-AUD and DFF-2-AUD films were significantly different than the others. Incompatibility such as this can lead to a reduction in tensile strength and elongation as stress points may result. Huang, Y., et al., J. Appl. Polym. Sci., 135 (32): 46542 (2018).

TABLE 1
Roughness values for the films.

| Sample | Glass side (SD) | | Air side (SD) | |
|---|---|---|---|---|
| Nonplasticised PVC | 5.8 nm | (1.7) | 73.9 nm | (15.0) |
| PVC plasticized with DEHP | 27.7 | (3.3) | 10.1 | (4.1) |
| PVC plasticized with THF-2-AUD | 9.8 | (0.7) | 25.3 | (7.1) |
| PVC plasticized with HMF-2-AUD | 137.3 | (57.3) | 114.9 | (41.9) |
| PVC plasticized with DFF-2-AUD | 37.2 | (14.7) | 139.3 | (118.6) |

The roughness of the films was determined in true non-contact mode using the SmartAnalysis software provided for the NX10 AFM system (Park Systems, Park Americas, Burlington, MA). These results are given in Table 1. There are three important take-aways from the roughness analysis. First, the roughness of the films varied depending on what was in direct contact with the films, glass or air. This suggests that having the same surface in contact with the films may influence a film's overall uniformity. Second, roughness analysis showed that the non-plasticized PVC film and those plasticized with DEHP or THF-2-AUD had comparable roughness, suggesting better miscibility. Third, roughness analysis suggests that although both HMF-2-AUD and DFF-2-AUD are less miscible with PVC (roughness at least twice that of PVC), immiscibility of films plasticized with HMF-2-AUD was not affected by the contact surface.

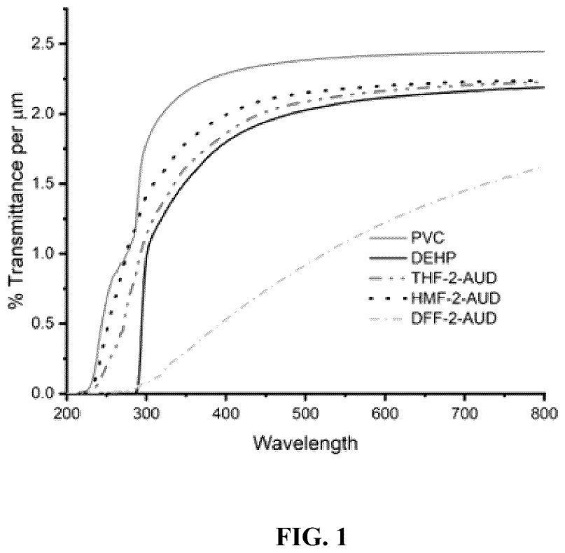

Light transmittance of the PVC films. The transmittance of the produced films, in terms of % transmission per micron of film thickness, was determined between 200 and 800 nm. The variability in this data is in general ±0.2% per micron. As can be seen in FIG. 1, the PVC control film is the most transparent. Below ˜375 nm, the amount of light passing through the PVC film begins to decline. The DEHP benchmark, however, has no transmittance below 300 nm due to the aromatic ring that is part of its structure. HMF-2-AUD and THF-2-AUD films had lower transmittance than the control, and their transmittance began to decrease below 450 nm. The transmittance of the DFF-2-AUD film was significantly lower than the others at all wavelengths. This is the result of our not being able to distil this plasticizer as we could with the others. With this reduction in transmission, the utility of this film in some packaging applications will be reduced. The HMF-2-AUD and THF-2-AUD would have similar value as the control plasticizer in providing films with similar clarity.

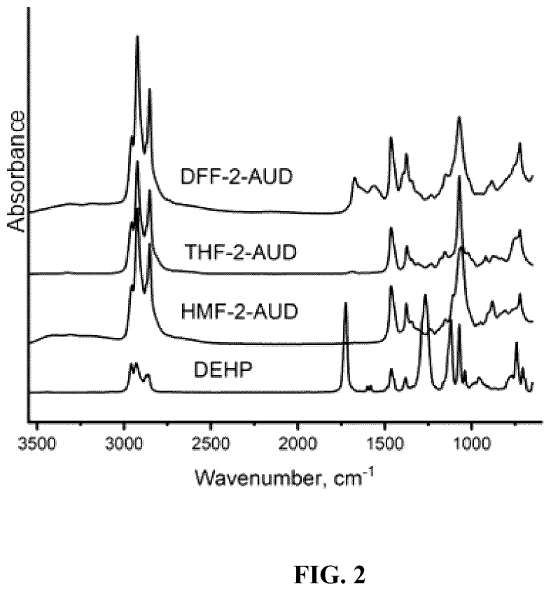

Infrared Spectra-Pure Plasticizers. The IR spectra (shown in FIG. 2) of the pure plasticizers were obtained between 600-3550 cm⁻¹. The pure HMF-2-AUD plasticizer displayed a weak broad peak at 3420 cm⁻¹, due to the O—H stretch resulting from the alcohol group at carbon 2. Relative to pure DEHP plasticizer, the pure THF-2-AUD, HMF-2-AUD, and DFF-2-AUD plasticizers had different absorbances in the aliphatic C—H stretch regions between 2850 and 2975 cm⁻¹. Given that DEHP has aromatic C—H bonds, these differences are to be expected. In the IR region from 775 to 1800 cm⁻¹, more differences were seen between the pure plasticizers. While the pure HMF-2-AUD and THF-2-AUD plasticizers were very similar, the pure DFF-2-AUD plasticizer displayed additional peaks at 1560, 1395, and 1150 cm⁻¹, as well as a strong peak at 1465 cm⁻¹ that can be attributed to the methylene group. As the DFF-2-AUD has an additional long carbon tail, such differences should be expected. The new plasticizers also have a strong band near 1070 cm⁻¹ that can be attributed to the C—O—C stretch of the furan ring. The DEHP, being an aromatic ester, was very different, having prominent peaks at 1725 and 1270, each attributable to the aromatic ester functionality.

### Plasticized PVC Films.

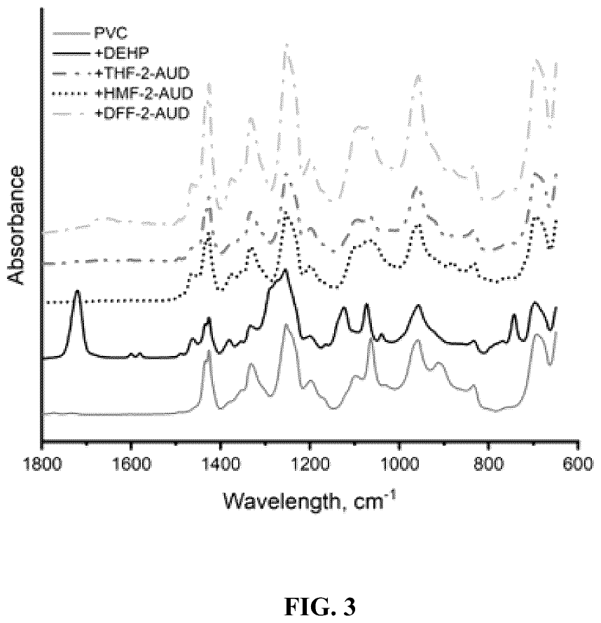

Infrared Spectra. FIG. 3 depicts FT-IR spectra from 650 to 1800 cm⁻¹ of the PVC control film, the PVC benchmark film with DEHP plasticizer, and the exemplary PVC films with THF-2-AUD, HMF-2-AUD, and DFF-2-AUD plasticizers, respectively.

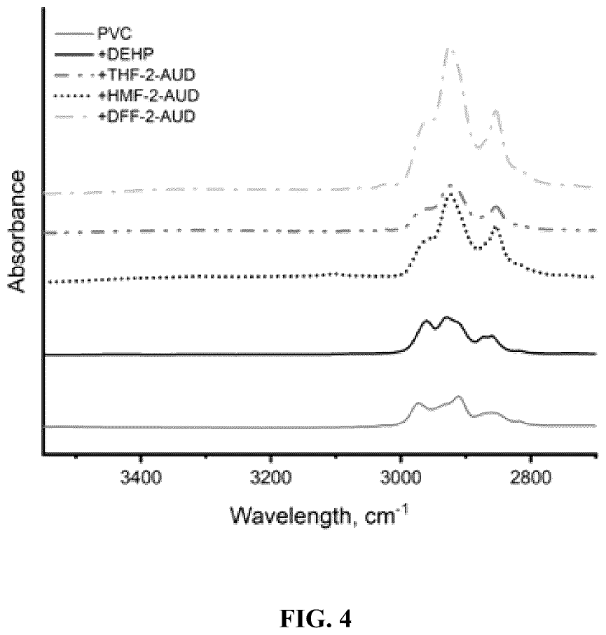

Additionally, the IR Spectra (shown in FIG. 4) for the PVC and plasticizer film formulations were obtained between 2700-3550 cm⁻¹. The spectra of the control and benchmark films made here match those detailed previously. Narita, S., et al., Journal of Polymer Science, 37 (131): 273-280 (1959) and Krimm, S., et al., Journal of Polymer Science Part A: General Papers, 1 (8): 2621-2650 (1963). All of the films were in general very similar to the control, especially the -2-AUD-containing films. With the addition of any plasticizers to the PVC, there was a small new peak at ˜1465 cm⁻¹ observed. In addition, incorporation of DEHP into the PVC film displays additional peaks at 1718, 1289, 1275, 1123, 1039, and 743 cm⁻¹. With the incorporation of DFF-2-AUD, a small peak at 1672 cm⁻¹ is present. For all of the PVC-plasticizer films, the additional peaks in the control film spectra are also observed in the pure plasticizers. This demonstrates that the IR spectra of the PVC/DEHP film is purely additive of the PVC and the plasticizer. It has been seen previously that when two materials interact, their IR spectra can change to a small degree. Max, J.-J., et al., The Journal of Chemical Physics, 119 (11): 5632-5643 (2003). For all film formulations, the peak at 909 cm⁻¹ in PVC shifts to ˜920 cm⁻¹. This peak has been attributed to the amorphous region of PVC. Krimm et al., ibid. Given that plasticizers will reside in the amorphous region of a polymer, changes here are reasonable. With changes in the plasticizer subtle changes in the IR spectra can be expected. The C—N stretch of the plasticizers made here are located at ˜1067 cm⁻¹ in the pure materials. When added to PVC, this peak undergoes a shift to ˜1102 cm⁻¹. For the spectra for the region from 2700 to 3550 cm⁻¹, it appears that the spectra of the PVC blended films are simple additions of the individual plasticizers and the PVC polymer.

TABLE 2
Tensile properties of PVC films with 22 wt % plasticizer.

| | Tensile strength | Elongation at | Young's |
|---|---|---|---|
| Plasticizer | (MPa) | break (%) | Modulus (MPa) |
| None | 54.3 ± 3.1^{a} | 105 ± 17^{d} | 732 ± 60^{a} |
| DEHP | 25.9 ± 1.6^{b} | 420 ± 31^{a} | 147 ± 17^{d} |
| THF-2-AUD | 28.9 ± 1.0^{c} | 340 ± 78^{b} | 317 ± 42^{b} |
| HMF-2-AUD | 26.4 ± 1.1^{b} | 429 ± 39^{a} | 270 ± 22^{c} |
| DFF-2-AUD | 26.4 ± 3.0^{b} | 191 ± 43^{c} | 360 ± 92^{b} |

Means without common letters differ significantly (P = 0.05) at a given Rh.

Tensile Properties. Upon the addition of plasticizers to a polymer, the tensile strength and Young's modulus typically decrease and the elongation increases. Boyer, R. F., J. Appl. Phys., 22 (6): 723-729 (1951). As shown in Table 2, with the addition of any of the plasticizers, a large reduction in tensile strength is seen. On average, tensile strength is reduced by 50% for each plasticizer, and the THF-2-AUD, HMF-2-AUD, and DFF-2-AUD plasticizers are at least equivalent to the benchmark DEHP plasticizer. As expected, the addition of the THF-2-AUD, HMF-2-AUD, and DFF-2-AUD plasticizers increases the elongation of the PVC blended films. The result of adding HMF-2-AUD is equivalent to DEHP in terms of elongation, whereas the increase in elongation is less when THF-2-AUD is added (i.e., less than that provided by DEHP or HMF-2-AUD). When DFF-2-AUD is used, the increase in elongation is much less. While all plasticizers decreased the modulus of the blended films, the DEHP gave the largest reduction followed by HMF-2-AUD. The modulus of the THF-2-AUD and DFF-2-AUD are higher than that of DEHP or HMF-2-AUD. Clearly, THF-2-AUD, HMF-2-AUD, and DFF-2-AUD function as plasticizers, however, the HMF-2-AUD provided superior performance relative to the THF-2-AUD and DFF-2-AUD plasticizers.

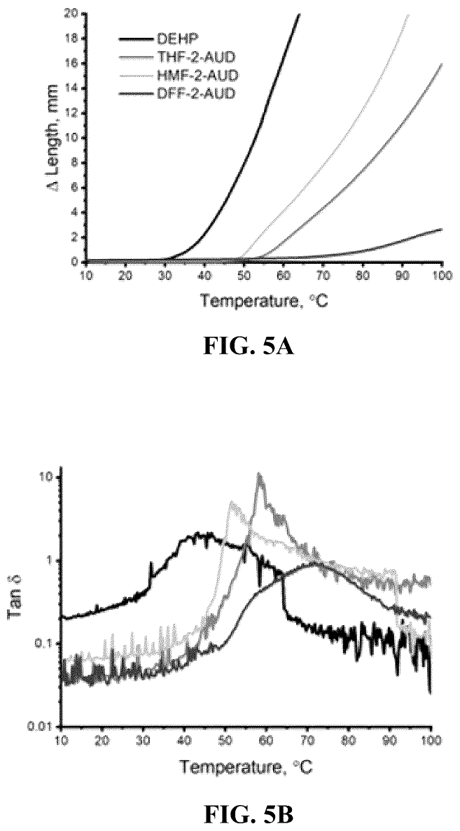

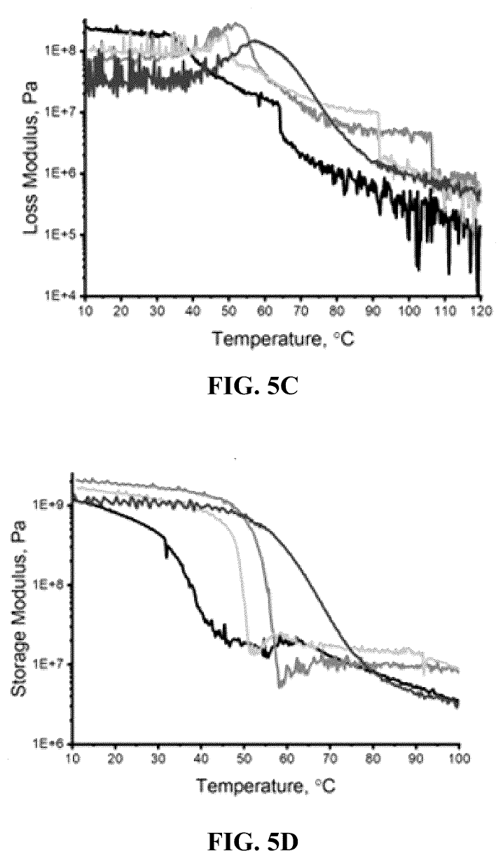

Dynamic Mechanical Analyses. The impact of heat on the physical properties of the PVC blended films was examined using dynamic mechanical analysis (DMA). FIGS. 5A-5D depict DMA curves for the PVC benchmark film with DEHP plasticizer and the exemplary PVC films with THF-2-AUD, HMF-2-AUD, and DFF-2-AUD plasticizers, respectively. FIG. 5A plots the change in length curve, FIG. 5B plots the tan 8 curve, FIG. 5C plots the loss modulus curve, and FIG. 5D plots the storage modulus curve. The key (legend) shown and used in FIG. 5A is also used (but not shown) in FIGS. 5B-5D. As the samples are heated, all films increased in length (FIG. 5A). The film containing DEHP increased in length by 1 mm at a lower temperature (36° C.) than did the films made with the THF-2-AUD, HMF-2-AUD, and DFF-2-AUD plasticizers, which increased in length at temperatures ranging from 51 to 83° C. Generally, when plasticizers are employed, the glass transition temperature, Tg, of the PVC is decreased. Scandola, M., et al., Polymer Bulletin, 6 (11): 653-660 (1982) and Hagen, R., et al., Polym. Test., 13 (2): 113-128 (1994). With this reduction in Tg, the impact that temperature has on the polymer's properties are magnified. Relative to DEHP, the new plasticizers undergo a smaller change in length at the same temperature. The DFF-2-AUD containing film sample underwent the least amount of sample extension and was, in fact, not very different from PVC. This suggests that the THF-2-AUD, HMF-2-AUD, and DFF-2-AUD plasticizers fail to match the benchmark DEHP as plasticizers for PVC. With heating, all PVC samples lose both storage and loss modulus. As observed with the change in length, the DEHP sample lost 20% of its storage or loss modulus at a lower temperature than the other plasticizers. Again, the HMF-2-AUD and THF-2-AUD samples began to lose modulus at a somewhat higher temperature. The DFF-2-AUD undergoes a loss in modulus at the highest temperature indicating that this PVC blend remains viscous and would resist flow at all but elevated temperatures. These results suggest again that the THF-2-AUD, HMF-2-AUD, and DFF-2-AUD plasticizers fail to match the benchmark DEHP as plasticizers for PVC. The ratio of loss modulus to storage modulus, termed Tan 8, can be used to define the glass transition temperature (Tg) of a polymer or polymer blend where the apex of the Tan 8 curve is the Tg. Selling, G. W., et al., Industrial Crops and Products, 178:114615 (2022). The incorporation of plasticizers increases the free volume of a polymer, which results in a reduction in the Tg. The more effective a plasticizer, the greater the reduction in Tg. The Tg of the DEHP film is lower than that of the THF-2-AUD, HMF-2-AUD, and DFF-2-AUD plasticizers, and DFF-2-AUD has the highest Tg. The use of all of these plasticizers results in a lower Tg than pure PVC. Rogalsky et al., ibid.

TABLE 3
Characteristic temperatures determined from the
dynamic mechanical analyses for PVC and plasticized
films prepared with each of the plasticizers.

| | Change in | Loss | Storage | Tan δ,^{c} |
|---|---|---|---|---|
| | Length,^{a} ° C. | modulus,^{b} ° C. | modulus,^{b} ° C. | ° C. |
| PVC | 80 | 92 | 86 | 85 |
| DEHP | 36 | 40 | 20 | 45 |
| THF-2-AUD | 57 | 57 | 50 | 59 |
| HMF-2-AUD | 51 | 52 | 41 | 51 |
| DFF-2-AUD | 83 | 64 | 52 | 71 |

^{a}Temperature where the length increased by 1 mm.
^{b}Temperature where modulus fell by 20%.
^{c}Temperature of peak Tan δ, or Tg.

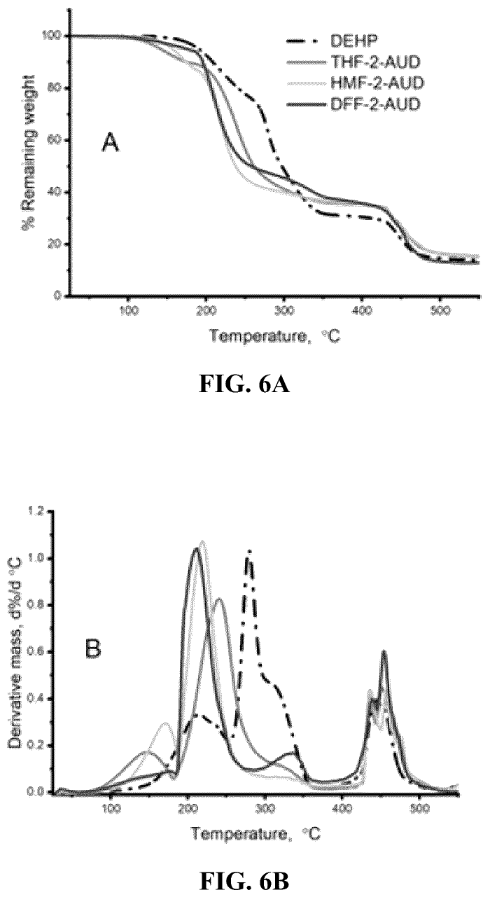

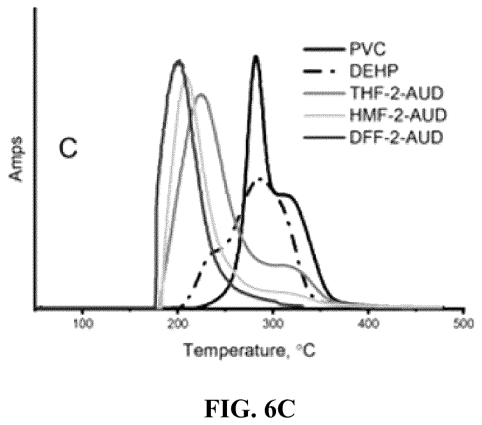

Thermogravimetric analyses. Of the five largest polymers produced, including PVC, polypropylene, polyethylene, polystyrene, and polyethylene terephthalate (PET), PVC has the lowest onset temperature for thermal degradation as measured by thermogravimetric analysis (TGA). Yu, J., et al., Waste Management, 48:300-314 (2016). Thermal degradation of PVC has an initial onset at about 250° C. and 65% weight loss by 350° C. resulting in liberated HCl. Given this, determining how the THF-2-AUD, HMF-2-AUD, and DFF-2-AUD plasticizers impact PVC degradation has importance. FIGS. 6A-6C depict TGA curves for the PVC benchmark film with DEHP plasticizer and the exemplary PVC films with THF-2-AUD, HMF-2-AUD, and DFF-2-AUD plasticizers, respectively. FIG. 6A plots the Mass Loss curve, FIG. 6B plots the First Derivative Mass Loss curve, and FIG. 6C plots the HCl Evolution curve. The key (legend) shown and used in FIG. 6A is also used (but not shown) in FIG. 6B; whereas, a slightly different key (legend) is shown and used in FIG. 6C that adds a thick black line to denote the HCl evolution curve for the PVC control film (i.e., without plasticizer). The temperature at which 10% mass loss has occurred for the four PVC blends were 209° C. for DEHP, 175° C. for THF-2-AUD, 174° C. for HMF-2-AUD, and 196° C. for DFF-2-AUD. The maximum rate of thermal degradation, as defined by the highest value for the first derivative of mass versus temperature, for the four PVC blends were 280° C. for DEHP, 241° C. for THF-2-AUD, 218° C. for HMF-2-AUD, and 212° C. for DFF-2-AUD. The results determined here for PVC plasticized with DEHP agree well with those determined previously. Halloran et al., ibid. The THF-2-AUD, HMF-2-AUD, and DFF-2-AUD plasticizers produced here all had a lower thermal degradation temperature, and a lower temperature for maximum weight loss, when compared to the industry standard DEHP. Keeping in mind that plasticizers are employed to improve product processing and value in end use (Umemura, R. T., et al., Journal of Applied Polymer Science, 138 (10): 49990 (2021)) and are known to affect PVC degradation (Wypych, G., Chapter 4-Principles of Thermal Degradation, in PVC Degradation and Stabilization (Third Edition), Wypych, G., Ed., ChemTec Publishing, 79-165 (2015)), we investigated PVC degradation of the films produced here (i.e., the PVC control film, the PVC benchmark film, and the PVC films plasticized with THF-2-AUD, HMF-2-AUD, and DFF-2-AUD, respectively). PVC degradation is a complex process that involves the loss of HCl. With this in mind, we also examined the evolution of HCl during degradation by using temperature programmed decomposition. These results correlate well with the TGA results. The temperatures at which the onset of HCl evolution occurs were found to be 225° C., 200° C., 180° C., 183° C., and 175° C. for PVC, and PVC plasticized with DEHP, THF-2-AUD, HMF-2-AUD, and DFF-2-AUD, respectively. These gas evolution traces are shown in FIG. 6C. In FIG. 6C, the HCl evolution curve for the PVC control film (i.e., without plasticizer) is depicted with a thick black line. HCl evolution from PVC during thermal composition was monitored at both m e 36 and 38, but only m/e 36 is shown in FIG. 6C for clarity. We also monitored masses that might indicate decomposition of the plasticizers. These included m/z 28 for CO or ethylene, 30 for ethane, 44 for CO₂ and 70 for Cl₂. Only trace amounts of these were found (not shown). The THF-2-AUD, HMF-2-AUD, and DFF-2-AUD plasticizers produced here all have basic amine functionality and their impact on thermal degradation was anticipated. Wypych, ibid. While this increased thermal degradation does impact the overall value of PVC blended articles in certain end-uses, these biobased plasticizers may have value in other roles.

Leachability of the plasticizers from PVC films. The loss of plasticizer from the polymer leads to product failure and the health and environmental issues associated with the additives. To this end, we examined the leachability of THF-2-AUD and HMF-2-AUD in water, 50% aqueous ethanol, and petroleum ether by soaking films for 48 h. These results are shown in Table 4 and are in agreement with what is suggested in SEM and AFM images (not shown): The apparent low compatibility of HMF-2-AUD with PVC leads to high leachability of this plasticizer. In fact, the 21.2% weight loss of HMF-2-AUD in petroleum ether is almost complete extraction of the plasticizer.

TABLE 4
Leachability of THF-2-AUD and HMF-
2-AUD plasticizer from PVC film.

| | | % Weight loss | |
|---|---|---|---|
| | | after 48 h soak | Petroleum |
| Plasticizer | H₂O | 50% Ethanol | ether |
| THF-2-AUD | 8.6 | 2.4 | 11.1 |
| HMF-2-AUD | 10.7 | 16.0 | 21.2 |

### Conclusions

The THF-2-AUD, HMF-2-AUD, and DFF-2-AUD compounds described in this paper effectively plasticize PVC, although none matched the benchmark plasticizer DEHP in all respects.

### Experimental

### Materials

The 2-undecanone, furfural, 2-(hydroxymethyl) furan, and poly(vinylchloride) were from MilliporeSigma. NH₃ (99.5%) was from Airgas, 5 wt % Pd/C was from Alfa Aesar and solvents were from Fischer Scientific.

Preparation of 2-aminoundecane. 2-undecanone was converted to 2-aminoundecane using a reductive amination. In a 2 L high-pressure reactor (Parr Instruments, Inc.) with a glass liner, 150 g 2-undecanone was dissolved in 1000 ml methanol along with 2 g 5 wt % Pd/C. The closed reactor was purged of air with stirring using argon and then charged with 1.7 bar ammonia followed by 34 bar H₂. The reactor was then heated to 80° C. Reaction progress was followed by GCMS using a Shimadzu QP2010 SE GC/mass spectrometer. Separations were accomplished using a Supelco Petrocol DH 50.2 (50 m×0.2 mm×0.5 μm) column. The oven program had an initial temperature 50° C. that was held for 3 min, followed by a ramp of 20° C./min to 275° C. with a final hold time of 2 min. The mass spectrometer was operated in the EI mode at 70 eV. Once the reaction was complete, the catalyst was removed by filtration and the filtrate was taken to dryness using a rotary evaporator. The final product was collected by vacuum distillation, 84° C./150 mtorr. GCMS 70 eV m/z (%) 44 (100), 41 (5.3), 43 (4.6). ¹H NMR (500 MHz, CDCl₃) δ 2.9 (m, 1H), 1.27 (broad s, 18H), 1.05 (d, J=6.5 Hz 3H), 0.9 (t J=7 Hz 3H). ¹³C NMR (125 MHz, CDCl₃) δ 46.9, 40.3, 31.9, 29.5, 26.4, 24.0, 22.7, 14.1.

Preparation of Diformylfuran, DFF. In a CEM microwave reactor with gas addition capabilities, 1.5 g HMF, 5 g α-MnO₂, and 45 ml acetonitrile were heated to 120° C. under 6 bar air. Reaction progress was followed by GCMS using the same instrument and heating profile as used for the 2-AUD synthesis. At completion, typically 2-4 h, the catalyst was removed by filtration and the DFF was recovered by rotary evaporation of the solvent.

General methods for the preparation of plasticizers. In a 300 mL high-pressure reactor (Parr Instruments, Inc.), 5.3 g (3.1 mmol) 2-aminoundecane and 3.1 mmol of the furan were dissolved in 40 ml methanol. To this was added about 300 mg 5 wt % Pd/C. This is a catalyst loading of 5 mol %. The closed reactor was then purged with H₂, heated to 80° C., and finally charged to 34 bar H₂. Reaction progress was followed by GCMS using the same instrument and heating profile described above. Upon completion of the reaction, the catalyst was removed by filtration and the solvent was removed by rotary evaporation. THF-2-AUD and HMF-2-AUD were collected by short path vacuum distillation though DFF-2-AUD could not be distilled.

N-((tetrahydrofuran-2-yl)methyl) undecane-2-amine (THF-2-AUD). ¹H NMR (500 MHZ, D₂O) δ 3.8 (dd, 1H, J=2.6 Hz, J=12 Hz), 3.5 (dm, 1H), 2.8 (dt, 1H), 2.65 (m, 2H), 1.9 (m, 3H), 1.7 (m, 2H), 1.3 (s, 14H), 1.07 (m, 3H), 0.9 (t, J=7 Hz, 3H). ¹³C NMR (125 MHz, D₂O) δ 80.3, 79.3, 78.6, 65.8, 64.8, 53, 37, 36.5, 31.9, 29, 27, 26, 22, 19.8, 14.1. MALDI-TOF MS: m/z 258, [M+Na]⁺. GCMS 70 eV m/z (%) 128 (100), 58 (56.6), 184 (53.8).

(5-((undecane-2-ylamino)tetrahydrofuran-2-yl) methanol (HMF-2-AUD) ¹H NMR (500 MHZ, D₂O) δ 4.0 (m, 1H), 3.8 (m, 1H), 3.7 (m, 1H), 2.75 (dd, J=3.7 Hz, J=12 Hz, 1H), 2.6 (m, 3H), 1.94 (dm, 3H), 1.5 (dm, 2H), 1.3 (broad s, 18H), 1.05 (dd, J=6 Hz, J=6 Hz, 3H), 0.9 (t, J=7 Hz, 3H). ¹³C NMR (125 MHZ, D₂O) δ 78, 68, 53, 52, 37, 30, 29, 26, 25.7, 22.6, 20, 14.1. MALDI-TOF MS: m/z 308, [M+Na]⁺. GCMS 70 eV m/z (%) 158 (100), 184 (78.3), 58 (56.0).

N,N′-((tetrahydrofuran-2,5-diyl)bis(methylene))bis(undecane-2-amine) (DFF-2-AUD) ¹H NMR 2.8 (m, 4H), 1.9 (m, 1H), 1.5 (m, 2H), 1.3 (broad s, 16H), 1.05 (dd, J=6 Hz, J=6 Hz, 3H), 0.9 (t, J=7 Hz, 3H).). (500 MHZ, CDCl₃) δ ¹³C NMR (125 MHz, CDCl₃) δ 80.4, 53.6, 52.2, 51.8, 31.9, 29.6, 29.2, 26.0, 22.6, 19.9, 14.0. MALDI-TOF MS: m/z 439, [M+H]⁺.

Preparation of the PVC films. The films were prepared by casting tetrahydrofuran solutions of poly(vinyl) chloride (average MW 62,000 Da) onto glass Petri dishes. Typically, 200 mg PVC were stirred in 5 ml THF until a homogenous solution was obtained. For plasticized films, 32 mg of the plasticizer were added to this solution. The cast films were allowed to dry in a fume hood for 24 h and were then further dried in a vacuum oven at ambient temperature for 48 h.

All of the references cited herein, including U.S. Patents and U.S. Patent Application Publications, are incorporated by reference in their entirety. Also incorporated by reference in their entirety are the following references: U.S. Pat. Nos. 8,541,626 and 11,192,913.

Thus, in view of the above, there is described (in part) the following:

A plasticizer of the formula:

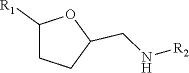

wherein R₁ is selected from the group consisting of a hydrogen atom,-MeOH, and an amine of the formula —C—NH—R₂, wherein each R₂ is independently an alkyl moiety derived from any ketone of the formula R₃—C(O)—R₃, wherein each R₃ is independently C1 to C22 straight or branched chain hydrocarbon which may be saturated or unsaturated.

A composition comprising (or consisting essentially of, or consisting of): at least one plasticizer of the formula:

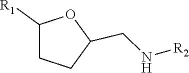

wherein R₁ is selected from the group consisting of a hydrogen atom,-MeOH, and an amine of the formula —C—NH—R₂, wherein each R₂ is independently an alkyl moiety derived from any ketone of the formula R₃—C(O)—R₃, wherein each R₃ is independently C1 to C22 straight or branched chain hydrocarbon which may be saturated or unsaturated; and optionally at least one polymer.

A method of making a plasticizer of the formula:

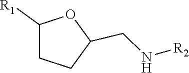

wherein R₁ is selected from the group consisting of a hydrogen atom,-MeOH, and an amine of the formula —C—NH—R₂, wherein each R₂ is independently an alkyl moiety derived from any ketone of the formula R₃—C(O)—R₃, wherein each R₃ is independently C1 to C22 straight or branched chain hydrocarbon which may be saturated or unsaturated, the method comprising:
(1) reacting an alkyl moiety derived from any ketone of the formula R₃—C(O)—R₃ with a catalyst, ammonia, and an organic solvent to form an amine of the formula R₃—NH₂—R₃, wherein each R₃ is independently C1 to C22 straight or branched chain hydrocarbon which may be saturated or unsaturated; and reacting the amine of the formula R₃—NH₂—R₃ with a catalyst, at least one furan derivative, and an organic solvent to form the plasticizer.

The term “consisting essentially of” excludes additional method (or process) steps or composition components that substantially interfere with the intended activity of the method (or process) or composition, and can be readily determined by those skilled in the art (for example, from a consideration of this specification or practice of the invention disclosed herein). The invention illustratively disclosed herein suitably may be practiced in the absence of any element (e.g., method (or process) steps or composition components) which is not specifically disclosed herein. Thus, the specification includes disclosure by silence (“Negative Limitations In Patent Claims,” AIPLA Quarterly Journal, Tom Brody, 41 (1): 46-47 (2013): “ . . . . Written support for a negative limitation may also be argued through the absence of the excluded element in the specification, known as disclosure by silence. Silence in the specification may be used to establish written description support for a negative limitation. As an example, in Ex parte Lin [No. 2009-0486, at 2, 6 (B.P.A.I. May 7, 2009)] the negative limitation was added by amendment. . . . In other words, the inventor argued an example that passively complied with the requirements of the negative limitation . . . was sufficient to provide support. . . . This case shows that written description support for a negative limitation can be found by one or more disclosures of an embodiment that obeys what is required by the negative limitation . . . .”

Other embodiments of the invention will be apparent to those skilled in the art from a consideration of this specification or practice of the invention disclosed herein. It is intended that the specification and examples be considered as exemplary only, with the true scope and spirit of the invention being indicated by the following claims.

What is claimed is:

1. A plasticizer of the formula: 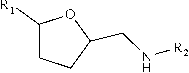
wherein R₁ is selected from the group consisting of a hydrogen atom,-MeOH, and an amine of the formula —C—NH—R₂, wherein each R₂ is independently an alkyl moiety derived from any ketone of the formula R₃—C(O)—R₃, wherein each R₃ is independently C1 to C22 straight or branched chain hydrocarbon which may be saturated or unsaturated.

2. The plasticizer of claim 1, wherein the plasticizer is of the formula:

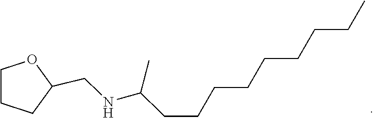 [SMILES: CCCCCCCCCC(C)NCC1CCCO1]

3. The plasticizer of claim 1, wherein the plasticizer is of the formula:

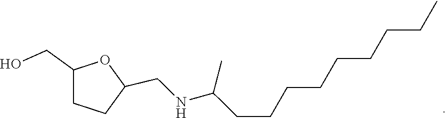 [SMILES: CCCCCCCCCC(C)NCC1CCC(CO)O1]

4. The plasticizer of claim 1, wherein the plasticizer is of the formula:

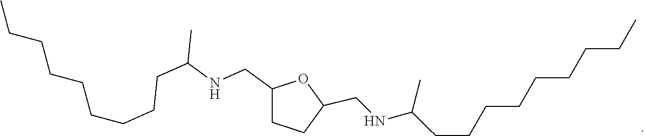 [SMILES: CCCCCCCCCC(C)NCC1CCC(CNC(C)CCCCCCCCC)O1]

5. The plasticizer of claim 1, wherein the alkyl moiety from which R₂ is derived is a crop-based ketone.

6. A composition comprising:
at least one plasticizer of the formula: 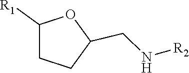
wherein R₁ is selected from the group consisting of a hydrogen atom,-MeOH, and an amine of the formula —C—NH—R₂, wherein each R₂ is independently an alkyl moiety derived from any ketone of the formula R₃—C(O)—R₃, wherein each R₃ is independently C1 to C22 straight or branched chain hydrocarbon which may be saturated or unsaturated; and
optionally at least one polymer.

7. The composition of claim 6, wherein the at least one polymer is selected from the group consisting of polyvinyl chloride (PVC), polylactic acid (PLA), cellulose acetate, nitrocellulose, cellulose acetate butyrate (CAB), alkyd resins, acrylic resins, nylon, polystyrene, polyurethanes, ethyl cellulose, polyvinyl acetate phthalate (PVAP), cellulose acetate phthalate (CAP), nitrile butadiene rubber (NBR), polychloroprene, ethylene propylene diene monomer (EPDM) rubber, chlorinated polyethylene, polymers derived from epichlorohydrin, and combinations thereof.

8. The composition of claim 6, wherein the at least one polymer includes polyvinyl chloride (PVC).

9. The composition of claim 6, further comprising at least one additional plasticizer.

10. The composition of claim 6, wherein the plasticizer is of the formula:

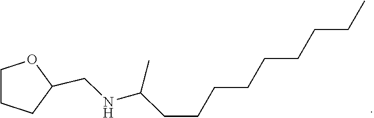 [SMILES: CCCCCCCCCC(C)NCC1CCCO1]

11. The composition of claim 6, wherein the plasticizer is of the formula:

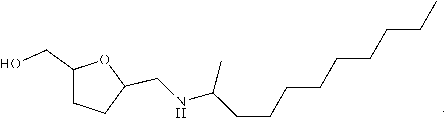 [SMILES: CCCCCCCCCC(C)NCC1CCC(CO)O1]

12. The composition of claim 6, wherein the plasticizer is of the formula:

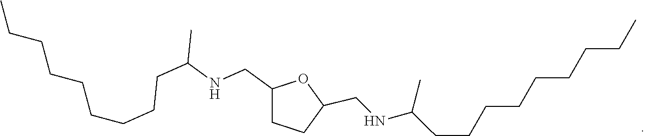 [SMILES: CCCCCCCCCC(C)NCC1CCC(CNC(C)CCCCCCCCC)O1]

13. A method of making a plasticizer of the formula: 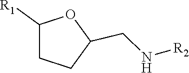
wherein R₁ is selected from the group consisting of a hydrogen atom,-MeOH, and an amine of the formula —C—NH—R₂, wherein each R₂ is independently an alkyl moiety derived from any ketone of the formula R₃—C(O)—R₃, wherein each R₃ is independently C1 to C22 straight or branched chain hydrocarbon which may be saturated or unsaturated, the method comprising:
(1) reacting an alkyl moiety derived from any ketone of the formula R₃—C(O)—R₃ with a catalyst, ammonia, and an organic solvent to form an amine of the formula R₃—NH₂—R₃, wherein each R₃ is independently C1 to C22 straight or branched chain hydrocarbon which may be saturated or unsaturated; and
(2) reacting the amine of the formula R₃—NH₂—R₃ with a catalyst, at least one furan derivative, and an organic solvent to form the plasticizer.

14. The method of claim 13, wherein the at least one furan derivative is selected from the group consisting of furfural, hydroxymethylfurfural (HMF), 2,5-diformylfuran (DFF), and combinations thereof.

15. The method of claim 13, wherein step (1) includes mixing the alkyl moiety derived from any ketone of the formula R₃—C(O)—R₃ and a molar excess of the ammonia in methanol as the organic solvent with the catalyst.

16. The method of claim 13, wherein step (2) includes mixing the at least one furan derivative in a freshly distilled state and a molar excess of the amine of the formula R₃—NH₂—R₃ in methanol as the organic solvent with the catalyst.

17. The method of claim 13, wherein step (1) includes mixing the alkyl moiety derived from any ketone of the formula R₃—C(O)—R₃ and approximately 10-25 fold molar excess of the ammonia in methanol as the organic solvent with a heterogeneous Pd/C catalyst as the catalyst.

18. The method of claim 13, wherein step (2) includes mixing the at least one furan derivative in a freshly distilled state and approximately 10% molar excess of the amine of the formula R₃—NH₂—R₃ in methanol as the organic solvent with a heterogeneous Pd/C catalyst as the catalyst.

19. The method of claim 13, wherein the catalyst in step (1) and/or step (2) is selected from the group consisting of palladium, platinum, rhodium, and combinations thereof.

20. The method of claim 13, wherein the catalyst in step (1) and/or step (2) is a heterogeneous palladium catalyst comprising 5 wt % palladium on carbon.
